(19) United States
(12) Patent Application Publication
(10) Pub. No.: US 2007/0118079 A1
(43) Pub. Date: May 24, 2007

(54) Medical devices and related systems and methods

(76) Inventors: John R. Moberg, Elk River, MN (US); Michael Gerdts, Big Lake, MN (US)

Correspondence Address: FISH & RICHARDSON PC, P.O. BOX 1022, MINNEAPOLIS, MN, 55440-1022, US

(21) Appl. No.: 11/284,718

(22) Filed: Nov. 21, 2005

(51) Int. Cl. A61M 5/178; A61M 31/00

(52) U.S. Cl. 604/164.07; 604/164.13; 604/510

(57) ABSTRACT

Medical devices and related systems and methods are described.

### TECHNICAL FIELD

This invention relates to medical devices and related methods.

### BACKGROUND

Systems are known for delivering medical devices, such as stents, into a body lumen. Often, such systems include a proximal portion that remains outside the body during use and a distal portion that is disposed within the body during use. The proximal portion typically includes a handle that is held by an operator of the system (e.g., a physician) during use, and the distal portion can include an outer tube surrounding an inner tube with a stent positioned between the inner and outer tubes. Generally, the operator of the system positions the distal portion within the lumen at a desired location. For example, the operator can position the distal portion so that the stent is adjacent an occlusion. The operator can then retract the outer tube to allow the stent to engage the occlusion and/or the lumen wall. After implanting the stent, the operator can remove the distal portion of the system from the lumen.

### SUMMARY

In general, the invention relates to systems that are configured to hold an elongate element (e.g., a guide wire) in a fixed position relative to an inner member (e.g., a catheter). The invention also relates to methods of using such systems. The systems can, for example, be used to treat an occluded body vessel (e.g., blood vessel) and/or to deliver an endoprosthesis (e.g., a stent) within the body vessel.

In one aspect of the invention, a system includes an inner member having a lumen that extends longitudinally through the inner member such that an elongate element (e.g., a guide wire) can be disposed within the lumen. A holding device is generally secured to the inner member (e.g., secured to a handle that is attached to the inner member) and includes a mechanism (e.g., a spring loaded member, an inflatable member, a clamp, a magnet) that is configured to hold the elongate element in a fixed position relative to the inner member. The inner member is generally surrounded by a retractable outer member (e.g., a sheath), and an endoprosthesis (e.g., a stent) is generally disposed between the inner member and the outer member.

To treat the occluded body vessel, the system can be inserted into the body vessel such that the portion of the inner member about which the endoprosthesis is located is positioned within the occluded region of the vessel. Upon being positioned as desired within the occluded region of the vessel, the holding device can be activated to fix the elongate element relative to the inner member. The outer member can then be retracted such that the endoprosthesis is delivered within the vessel.

In some embodiments, the system is configured to be switched between a first position in which the elongate element is substantially fixed relative to the inner member and a second position in which the elongate element is free to move relative to the member.

In certain embodiments, the inner member includes a first region that is more compliant than a second region. In such embodiments, the holding device can be configured to deform the first region such that the first region contacts the elongate element disposed in the lumen. The contact between the elongate element and the first region can fix the elongate element relative to the inner member.

In some embodiments, the holding device is configured to directly contact the elongate element. The contact between the holding device (e.g., a member of the holding device) and the elongate element can fix the elongate element relative to the inner member.

In certain embodiments, the holding device is configured to hold the elongate element in a fixed position relative to the inner member using magnetic force.

Embodiments may include one or more of the following advantages.

In certain embodiments, the elongate element is substantially prevented from moving relative to the inner member. Fixing the elongate element relative to the inner member can help to improve the precision with which a procedure can be performed within the body vessel. It can, for example, help to improve the precision with which the endoprosthesis can be deployed within the body vessel.

In some embodiments, the holding device is positioned near a handle (e.g., in the handle) of the system. This can help to enable the system to be operated by a single user (e.g., by a single physician).

In certain embodiments, a manually operable mechanism (e.g., a push button) is connected to the holding device. The manually operable mechanism can be manipulated (e.g., pushed) to activate the holding device in order to fix the elongate element relative to the inner member or to free the elongate element for movement relative to the inner member. In some embodiments, the holding device and the manually operable mechanism are disposed within the handle, which allows the user to activate the holding device while grasping the handle. In certain embodiments, the user can simultaneously grasp the handle and activate the holding device with a single hand. This can allow the user to perform other functions with his or her free hand, such as retracting the outer member.

In some embodiments, the manually operable mechanism can improve the user's control of the resistance provided to the elongate element. In certain embodiments, for example, tactile feedback may be provided to the hand of the user via the manually operable mechanism, which can help the user to determine whether a desirable amount of friction or resistance is being provided to the elongate element.

In certain embodiments, the holding device can be locked in at least one of a first position in which the elongate element is substantially fixed relative to the inner member and a second position in which the elongate element is free to move longitudinally relative to the inner member. The locking of the holding device can increase the efficiency with which the user can operate the system.

Other aspects, features, and advantages are in the description, the drawings, and the claims.

### DESCRIPTION OF DRAWINGS

FIG. 1 is a cross-sectional view of an embodiment of a medical system including a spring loaded member in a locked configuration.

FIG. 2 is a cross-sectional view of the medical system of FIG. 1 in an unlocked configuration.

FIGS. 3-5 illustrate an embodiment of a method of using the medical system of FIGS. 1 and 2.

FIG. 6 is a cross-sectional view of an embodiment of a medical system including an inflatable member in an unlocked configuration.

FIG. 7 is a cross-sectional view of the medical system of FIG. 6 in a locked configuration.

FIG. 8 is a cross-sectional view of an embodiment of a medical system including multiple inflatable members in an unlocked configuration.

FIG. 9 is a cross-sectional view of the medical system of FIG. 8 in a locked configuration.

FIG. 10 illustrates an embodiment of an inner member of a medical system.

FIG. 11 is a cross-sectional view of an embodiment of a medical system including an inflatable member at a distal portion of the system, the system being in a locked configuration.

FIG. 12 is a cross-sectional view of the medical system of FIG. 11 in an unlocked configuration.

FIG. 13 is a cross-sectional view of an embodiment of a medical system including a clamp in an unlocked configuration.

FIG. 14 is a cross-sectional view of the medical system of FIG. 13 in a locked configuration.

FIG. 15 is a cross-sectional view of an embodiment of a medical system including a magnet in a locked configuration.

FIG. 16 is a cross-sectional view of the medical system of FIG. 15 in an unlocked configuration.

FIG. 17 is a partial side view of an embodiment of a rapid exchange catheter system.

Like reference symbols in the various drawings indicate like elements.

### DETAILED DESCRIPTION

In general, systems include a member (e.g., a catheter) with a lumen extending through the member. The systems are generally configured so that, when an elongate element (e.g., a guide wire) is disposed within the lumen, the elongate element can be held in a substantially fixed position relative to the member. In some embodiments, the systems are configured to be switched between a first position in which the elongate element is substantially fixed relative to the member and a second position in which the elongate element is free to move relative to the member. Examples of such systems are described below.

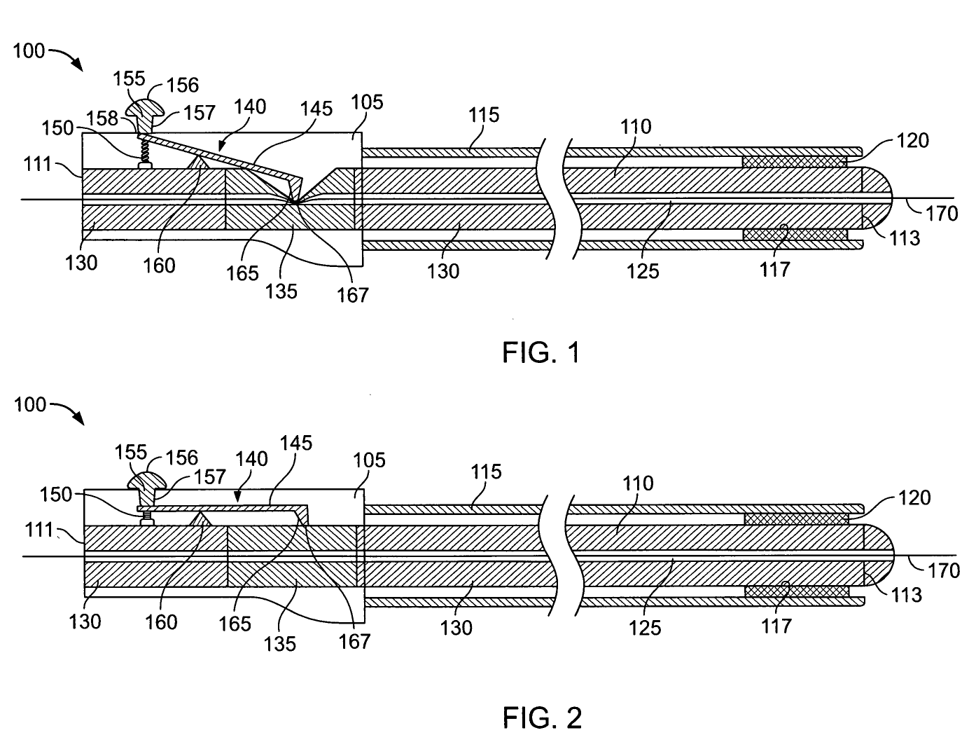

Referring to FIGS. 1 and 2, a system 100 includes an inner member (e.g., a catheter) 110 having a relatively compliant region 135 positioned between two relatively rigid regions 130. Relatively compliant region 135 and relatively rigid regions 130 can be attached to one another using any of various techniques, such as co-extrusion, thermal bonding, adhesive bonding, and/or mechanical attachments. A lumen 125 extends along the longitudinal axis of inner member 110 from a proximal end 111 of inner member 110 to a distal end 113 of inner member 110. An outer member (e.g., a sheath) 115 surrounds inner member 110 and extends to distal end 113 of inner member 110. An endoporosthesis (e.g., a self-expanding stent) 120 is disposed adjacent a distal region 117 of inner member 110 between inner member 110 and outer member 115. Outer member 115, while surrounding endoprosthesis 120, can prevent endoprosthesis 120 from expanding.

A handle 105 is secured to a proximal region of inner member 110. Handle 105 can, for example, be secured to inner member 110 using any of various attachment techniques, such as adhesive attachment techniques and/or mechanical attachment techniques.

Handle 105 includes a holding mechanism 140. Holding mechanism 140 can be operated to switch system 100 between a locked configuration (e.g., a configuration in which a guide wire 170 extending through lumen 125 is fixed relative to inner member 110) and an unlocked configuration (e.g., a configuration in which guide wire 170 extending through lumen 125 is free to move relative to inner member 110). Holding mechanism 140 includes a beam 145 that is free at its distal end and is attached to an activation button 155 at its proximal end. A contact member 165 extends from the distal end of beam 145. Contact member 165 includes a pointed tip 167. A spring 150 links button 155 to handle 105 (e.g., to an inner housing wall of handle 105). Spring 150 can, for example, be fixed at its bottom end to handle 105 and can be fixed at its top end to button 155. Button 155 is configured so that it can be moved in and out of handle 105. Button 155 includes a head 156 attached to a stem 157. Stem 157 is sized to fit through an aperture 158 formed in handle 105, while head 156 is larger than aperture 158 such that head 156 is prevented from passing through aperture 158 and remains outside of handle 105. Spring 150 can provide resistance to the inward and/or outward movement of button 155. As button 155 is actuated, it causes beam 145 to pivot about a fulcrum 160, which is fixed to handle 105 between the proximal and distal ends of beam 145.

When system 100 is in a locked position, as shown in FIG. 1, spring 150 is expanded, which forces button 155 and the proximal end of beam 145 outward (e.g., away from inner member 110). As a result, the distal end of beam 145 and contact member 165 are forced inward (e.g., toward inner member 110), causing contact member 165 to contact an outer surface of region 135. The rotational force applied to beam 145 by spring 150 causes contact member 165 to deform region 135 of inner member 110, causing the wall of region 135 to press against guide wire 170. The pressure applied to guide wire 170 by the wall of region 135 can help to reduce (e.g., prevent) the ability of guide wire 170 to move longitudinally within lumen 125.

When system 100 is in an unlocked position, as shown in FIG. 2, button 155 is depressed and spring 150 is compressed between button 155 and handle 105. As compared to the locked position of FIG. 1, the proximal end of beam 145 is positioned nearer the outer surface of inner member 110 and the distal end of beam 145 is positioned farther from the outer surface inner member 110 (e.g., out of contact with the outer surface of inner member 110). Consequently, region 135 of inner member 110 is substantially undeformed and guide wire 170 is free to move longitudinally within lumen 125.

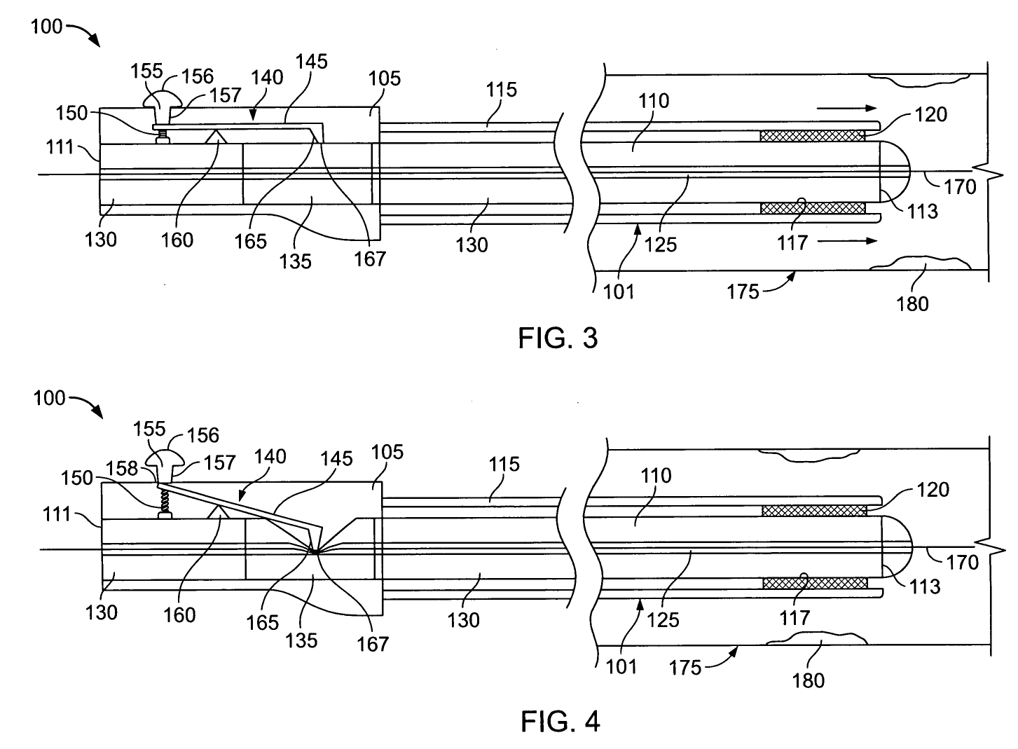

FIGS. 3-5 describe an exemplary method of using system 100. Referring to FIG. 3, guide wire 170 can be inserted into a body vessel (e.g., a blood vessel) 175 of a subject. A distal portion 101 of system 100 can then be inserted over guide wire 170 into body vessel 175. While inserting distal portion 101 of system 100 into body vessel 175, the user can depress button 155 so that inner member 110 is free to slide over guide wire 170 without substantial resistance. The user can, for example, depress button 155 with a finger or thumb of the hand in which handle 105 is grasped. Guide wire 170 can help to track or guide distal portion 101 of system 100 as it is pushed through body vessel 175.

The user can continue to insert distal portion 101 of system 100 into body vessel 175 until distal region 117 of inner member 110 (e.g., the region about which endoprosthesis 120 is located) is positioned within a target region (e.g., an occluded region) 180 of body vessel 175. Once distal region 117 of inner member 110 is positioned within target region 180 of body vessel 175, as shown in FIG. 4, the user can release button 155 to place system 100 in the locked position such that guide wire 170 becomes fixed relative to inner member 110.

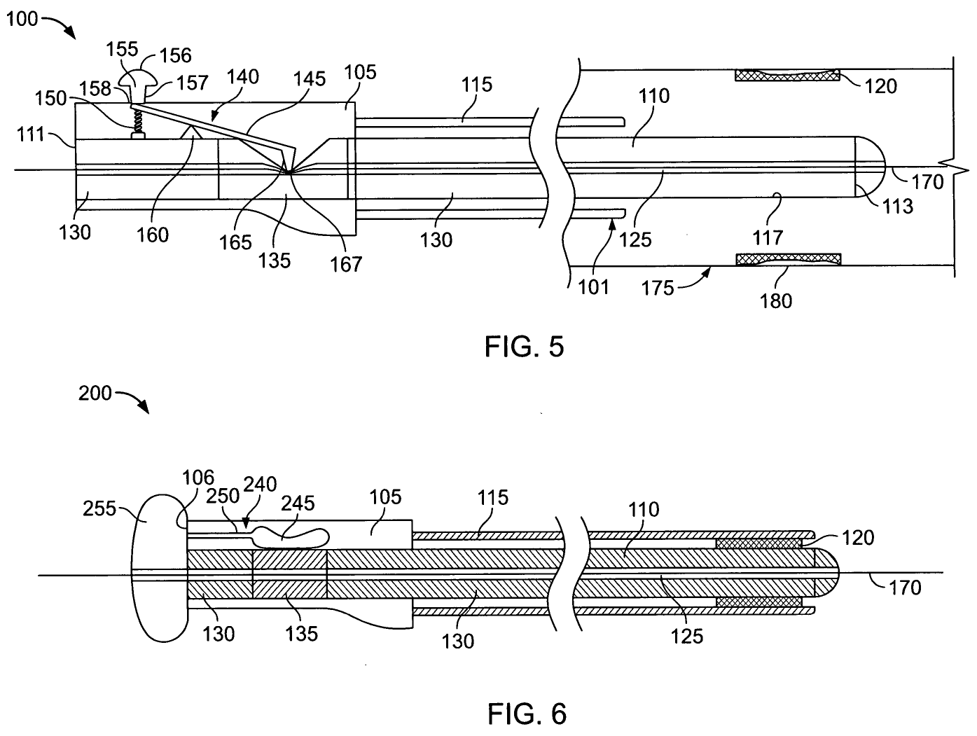

Referring to FIG. 5, after fixing guide wire 170 relative to inner member 110, the user can retract outer member 115 to deploy endoprosthesis 120. Because guide wire 170 is fixed relative to inner member 110 while deploying endoprosthesis 120, proximal and/or distal movement of inner member 110 resulting from the retraction of outer member 115 can be reduced (e.g., substantially prevented). It is believed that friction between inner member 110 and guide wire 170 in regions distal to the point at which region 135 is pressed against guide wire 170 can prevent reactionary forces associated with retraction of outer member 115 and/or potentional energy associated with any slack within inner member 110 from moving inner member 110 relative to guide wire 170. Consequently, it is believed that the precision with which endoprosthesis 120 can be deployed within body vessel 175 can be improved.

After deploying endoprosthesis 120 within body vessel 175, distal portion 101 of system 100 can be removed from body vessel 175. System 100 can be held in an unlocked position while removing distal portion 101 from body vessel 175 so that guide wire 170 can remain within body vessel 175 as distal portion 101 is removed. Alternatively, system 100 can be held in a locked position while removing distal portion 101 from body vessel 175 so that guide wire 170 can be simultaneously removed from body vessel 175.

Relatively rigid regions 130 are generally less compliant than region 135. In certain embodiments, regions 130 are harder than region 135. Regions 130 can, for example, have a hardness of about 60 D to about 90 D (e.g., about 70 D to about 80 D), and region 135 can have a hardness of about 50 A to about 70 A (e.g., about 55 A to about 65 A).

In some embodiments, the wall of region 135 is thinner than the walls of regions 130. The wall of region 135 can, for example, be about 0.005 inch to about 0.010 inch (about 0.127 millimeter to about 0.254 millimeter) thinner than the walls of regions 130. In some embodiments, the wall of region 135 has a thickness of about 0.004 inch to about 0.008 inch (about 0.102 millimeter to about 0.203 millimeter). The thinness of the wall of region 135 relative to the wall of regions 130 can help to make region 135 more compliant than regions 130.

Regions 130 can include one or more materials that are capable of withstanding forces associated with inserting inner member 110 into body vessel 175. In some embodiments, regions 130 include one or more polyamides (e.g., Vestamid®), polyetheretherketones (PEEKs), polyether block amides (e.g., Pebax®), polytetrafluoroethylenes (e.g., Teflon®), polyether-block co-polyamide polymers (e.g., Pebax®), copolyester elastomers (e.g., Arnitel® copolyester elastomers), thermoplastic polyester elastomers (e.g., Hytrel®), thermoplastic polyurethane elastomers (e.g., Pellethane™), polyeolefins (e.g., Marlex® polyethylene, Marlex® polypropylene), high-density polyethylenes (HDPEs), low-density polyethylenes (LDPEs), and/or polyimides.

Region 135 can include one or more resilient materials. In certain embodiments, region 135 includes one or more silicones, polyurethane elastomers (e.g., Pellethane®), thermoplastic rubbers (e.g., Alcryn®), rubbers, styrenic block copolymers (SBCs) (e.g., Kraton®), and urethanes.

Beam 145 of holding device 140 can be formed of one or more materials that are capable of deforming region 135. Beam 145 is generally less compliant than region 135 so that beam 145 can deform region 135 without becoming substantially deformed itself. Examples of materials from which beam 145 can be formed include stainless steel, thermoplastic, and thermoset materials.

Spring 150 can include any of various types of springs, such as compression springs, coil springs, and leaf springs. In some embodiments, spring 150 has a spring force of about two pounds or greater (e.g., about three pounds or greater, about four pounds or greater) and/or about five pounds or less (e.g., about four pounds or less, about three pounds or less). Spring 150 can include one or more materials, such as steel (e.g., stainless steel) and/or plastic.

As an alternative or in addition to using a spring to apply a force to button 155 and beam 145, other types of devices can be used. For example, elastic bands can be used to elastically connect beam 145 to inner member 110 (e.g., to compliant region 135 of inner member 110).

Handle 105 can be formed of one or more relatively rigid materials. The rigidity of handle 105 can help to provide a stable structure to which one or more of the components of holding device 140 (e.g., spring 150 and fulcrum 160 of holding device 140) can be supported. Examples of materials from which handle 105 can be formed include thermoplastics (e.g., Acrylonitrile Butadiene Styrene (ABS), polycarbonate).

Endoprosthesis 120 is generally a self-expanding stent. Examples of materials from which endoprosthesis 120 can be formed include shape memory materials, such as nitinol, silver-cadmium (Ag—Cd), gold-cadmium (Au—Cd), gold-copper-zinc (Au—Cu—Zn), copper-aluminum-nickel (Cu—Al—Ni), copper-gold-zinc (Cu—Au—Zn), copper-zinc/(Cu—Zn), copper-zinc-aluminum (Cu—Zn—Al), copper-zinc-tin (Cu—Zn—Sn), copper-zinc-xenon (Cu—Zn—Xe), iron beryllium (Fe₃Be), iron platinum (Fe₃Pt), indium-thallium (In—Tl), iron-manganese (Fe—Mn), nickel-titanium-vanadium (Ni—Ti—V), iron-nickel-titanium-cobalt (Fe—Ni—Ti—Co) and copper-tin (Cu—Sn). For yet additional shape memory alloys, see, for example, Schetsky, L. McDonald, “Shape Memory Alloys”, Encyclopedia of Chemical Technology (3rd ed.), John Wiley & Sons, 1982, vol. 20. pp. 726-736.

Guide wire 170 can be formed of a relatively flexible material, which can help to allow guide wire 170 to be navigated through tortuous regions of vessels. Guide wire 170 can, for example, be formed of one or more metals (e.g., stainless steels, nitinol) and/or polymeric materials (e.g., polyamides, nylons). Examples of other materials from which guide wire 170 can be formed are described in U.S. Pat. No. 6,436,056 issued to Wang et al., which is incorporated by reference herein.

While embodiments of spring loaded holding devices have been described, other types of holding devices can alternatively or additionally be used. Referring to FIG. 6, for example, a system 200 includes a holding device 240 having an inflatable member (e.g., a balloon) 245 that is fluidly connected to a fluid pump 255. Inflatable member 245 is positioned adjacent region 135 of inner member 110. An inflation lumen 250 connects fluid pump 255 to inflatable member 245. Inflation lumen 250 can be integrally formed within handle 105. Alternatively or additionally, inflation lumen 245 can include tubing disposed within handle 105. Fluid pump 255 is positioned (e.g., attached) at a proximal end 106 of handle 105. The positioning of fluid pump 255 in close proximity to handle 105 can help to allow the user of system 200 to operate fluid pump 255 while grasping handle 105.

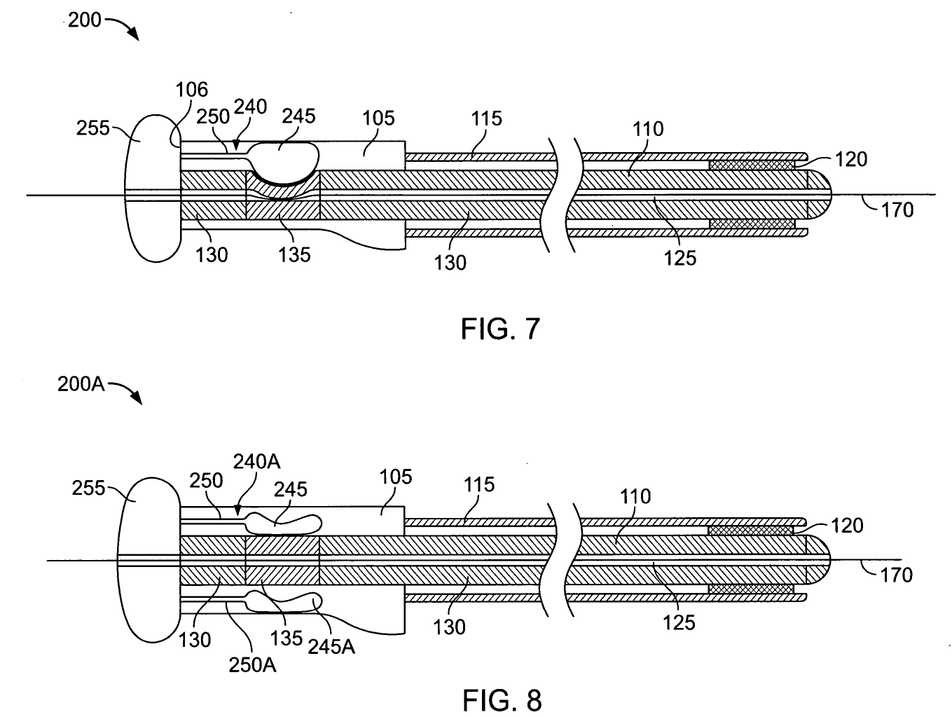

When inflatable member 245 is deflated, as shown in FIG. 6, region 135 is substantially undeformed and guide wire 170 is free to move longitudinally within lumen 125. In order to longitudinally fix guide wire 170 relative to inner member 110, inflatable member 245 can be inflated, as shown in FIG. 7. Inflatable member 245 can be inflated by pumping an inflation fluid (e.g., saline) from fluid pump 255 through inflation lumen 250 to inflatable member 245. The inflation of inflatable member 245 can deform region 135, which can compress guide wire 170 between opposite sides of region 135. The compression of guide wire 170 between opposite sides of region 135 can substantially prevent guide wire 170 from moving longitudinally within lumen 125.

Fluid pump 255 can include one or more devices capable of transferring the inflation fluid to inflatable member 245. Examples of such devices include syringes and biflators. In some embodiments, fluid pump 255 is manually operable. In certain embodiments, for example, fluid pump 255 can be actuated by depression of the user's thumb or finger, which can enable inflation of inflatable member 245 while simultaneously grasping handle 105.

While fluid pump 255 has been described as being positioned at proximal end 106 of handle 105, fluid pump 255 can alternatively or additionally be located at other positions. In certain embodiments, for example, fluid pump 255 is disposed within handle 105. In some embodiments, fluid pump 255 is detached from handle 105.

While inflatable member 245 has been described as a balloon, other types of inflatable members can alternatively or additionally be used. Examples of other types of inflatable members include inflatable cuffs.

Inflatable member 245 can be formed of one or more distensible materials. Examples of materials from which inflatable member 245 can be formed include polyurethanes and block copolymers, such as polyamide-polyether block copolymers or amide-tetramethylene glycol copolymers. Examples include the Pebax® (a polyamide/polyether/polyester block copolymer) family of polymers, e.g., Pebax® 70D, 72D, 2533, 5533, 6333, 7033, or 7233 (available from Elf AtoChem, Philadelphia, Pa.). Other examples include nylons, such as aliphatic nylons, for example, Vestamid L2101F, Nylon 11 (Elf Atochem), Nylon 6 (Allied Signal), Nylon 6/10 (BASF), Nylon 6/12 (Ashley Polymers), or Nylon 12. Additional examples of nylons include aromatic nylons, such as Grivory (EMS) and Nylon MXD-6. Other nylons and/or combinations of nylons can be used. Still other examples include polybutylene terephthalate (PBT), such as Celanex® (available from Ticona, Summit, N.J.), polyester/ether block copolymers such as Arnitel® (available from DSM, Erionspilla, Ind.), e.g., Arnitel® EM740, aromatic amides such as Trogamid (PA6-3-T, Degussa), and thermoplastic elastomers such as Hytrel® (Dupont de Nemours, Wilmington, Del.).

While the inflation fluid has been described as saline, other types of fluids can be used. Examples of inflation fluids include liquids (e.g., saline, contrast solution, water) and gases (e.g., inert gases, such as air).

While embodiments of holding devices have been described in which the holding device is configured to compress or deform region 135 at a single location (e.g., at the top of region 135), the holding device can alternatively or additionally be configured to compress or deform region 135 at multiple locations. The holding device can, for example, include multiple members that are spaced around the circumference of region 135. Alternatively or additionally, multiple members can be longitudinally spaced along region 135.

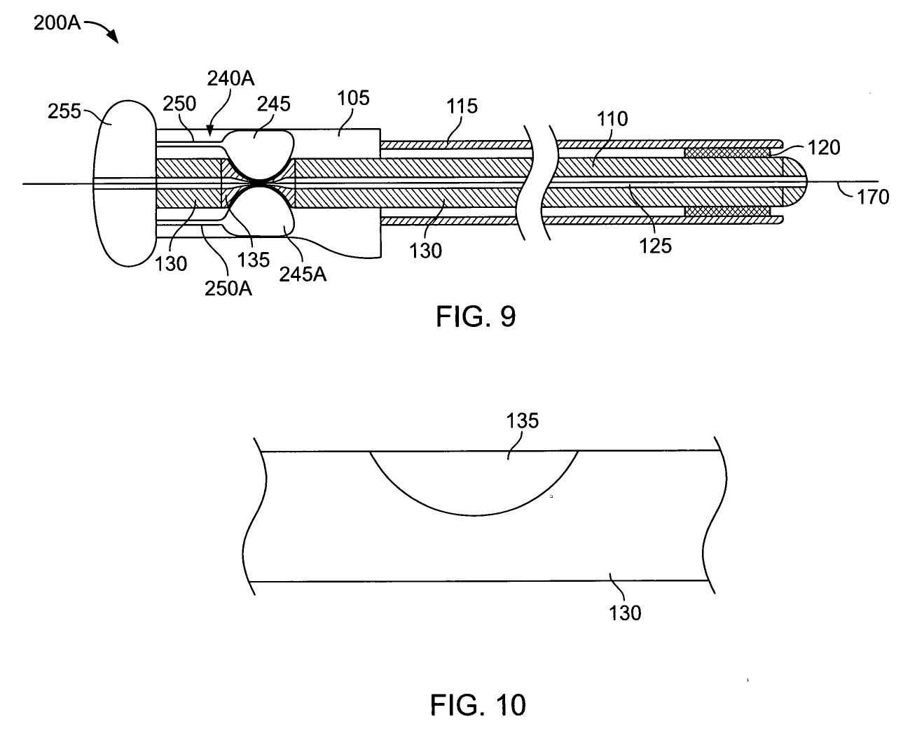

FIGS. 8 and 9 illustrate an example of a system 200A with a holding device 240A that includes multiple members 245 and 245A. As shown, inflatable members 245 and 245A are positioned on opposite sides of region 135. Similar to inflatable member 245, inflatable member 245A is fluidly connected to fluid pump 255 via an inflation lumen 250A. When inflatable members 245 and 245A are inflated, as shown in FIG. 9, guide wire 170 is compressed between opposite walls of region 135.

While various embodiments of holding devices have been described, other types of holding devices that are capable of deforming region 135 can alternatively or additionally be used. Examples of other types of holding devices include clamps, Touhy-Borst valves, and EAP rings.

While relatively compliant region 135 of the embodiments above extends around the entire circumference of the inner member, other configurations are possible. In some embodiments, region 135 extends about only a portion of the circumference of the inner member. As shown in FIG. 10, for example, region 135 extends along a top portion of the inner member. Consequently, a guide wire can be compressed between region 135 and the portion of relatively rigid region 130 opposite region 135. This arrangement can help to reduce (e.g., prevent) deformation of the opposite side wall of the inner member while region 135 is deformed by the holding device. Consequently, the force with which guide wire 170 is compressed within lumen 125 can be increased.

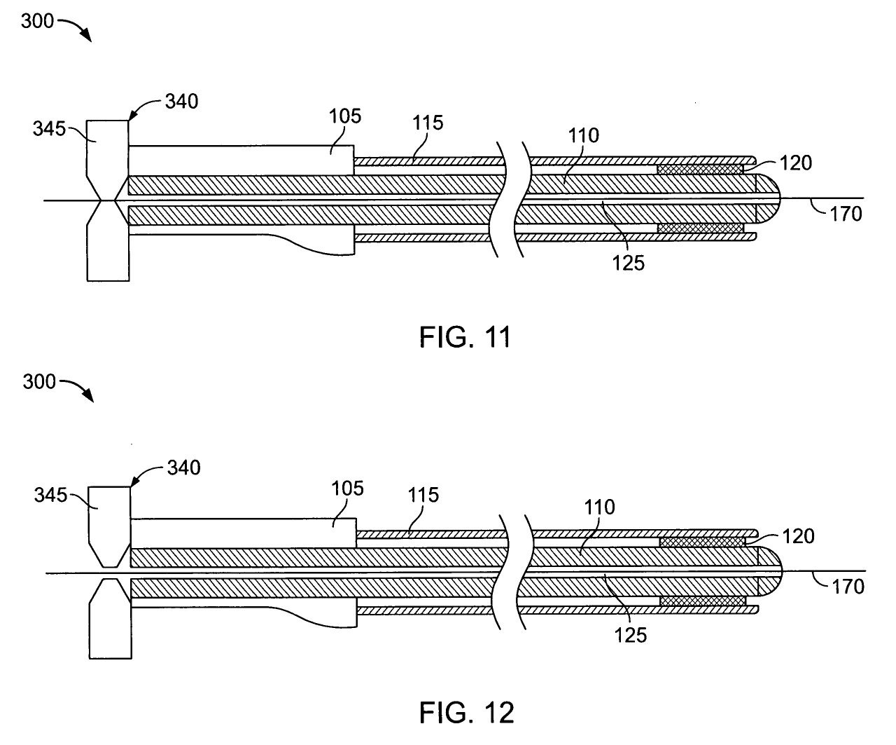

While the embodiments above describe deforming region 135 in order to secure guide wire 170 relative to inner member 110, other techniques can be used. In certain embodiments, for example, holding devices can be arranged to contact (e.g., compress) the guide wire directly. For example, as shown in FIGS. 11 and 12, a system 300 includes a holding device 340 positioned at the proximal end of handle 105. Holding device 340 includes a clamp 345 that can be secured to handle 105 using any of various techniques. Clamp 345 can, for example, be thermally bonded, adhesively bonded, and/or mechanically attached to handle 105.

During use, the user can activate holding device 340 so that clamp 345 compresses guide wire 170, as shown in FIG. 11. Consequently, guide wire 170 can be prevented from moving longitudinally relative to inner member 110. The user can also deactivate holding device 340 so that clamp 345 is released from contact with guide wire 170, as shown in FIG. 12. As a result, guide wire 170 is free to move longitudinally within lumen 125.

While holding device 340 has been described as including a clamp, it should be understood that any of the other types of holding devices described herein can alternatively or additionally be used to compress guide wire 170. For example, a spring loaded member and/or an inflatable member can similarly be arranged to contact guide wire 170 directly in order to hold guide wire longitudinally fix guide wire 170 relative to inner member 110.

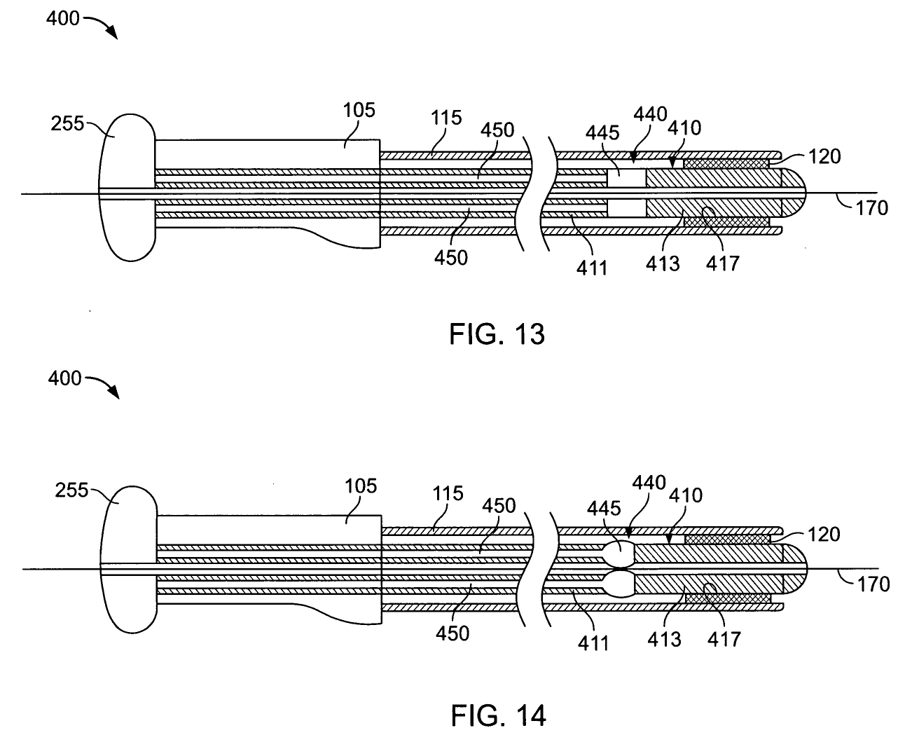

While the embodiments above describe holding devices that are positioned near the proximal end of the system (e.g., near handle 105), holding devices or members of holding devices can alternatively or additionally be positioned nearer the distal end of the system. Referring to FIGS. 13 and 14, for example, a system 400 includes a holding device 440, which includes a ring-shaped inflatable member 445 positioned near a distal region 417 of an inner member 410. Inflatable member 445 is positioned between a proximal portion 411 and a distal portion 413 of inner member 410. Inflatable member 445 can be joined to proximal and distal portions 411 and 413 using any of various techniques, such as thermal bonding, adhesive bonding, and/or mechanical attachment techniques. Inflatable member 445 is fluidly connected to fluid pump 255 by inflation lumens 450. Inflation lumens 450 can be formed within catheter 410. Alternatively or additionally, inflation lumens 450 can include one or more tubes extending along inner member 410. Upon inflating inflatable member 445, guide wire 170 is compressed between inner surfaces of inflatable member 445. This compressive force can help to prevent guide wire 170 from moving longitudinally relative to inner member 110.

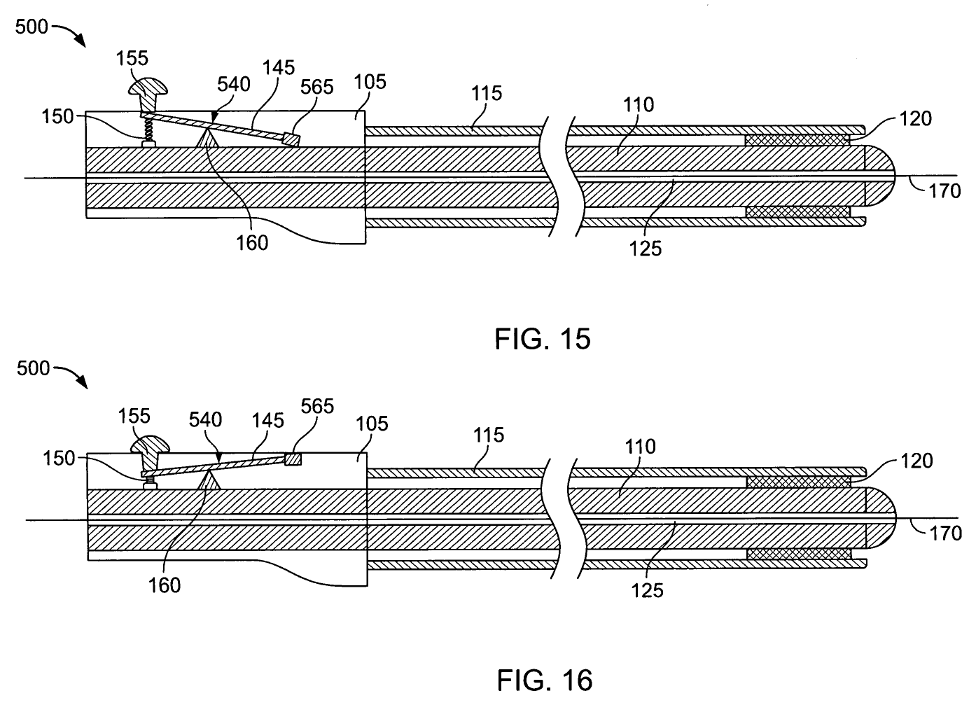

While the embodiments described above include holding devices that are configured to deform region 135 and/or compress guide wire 170, other types of holding devices can alternatively or additionally be used. Referring to FIGS. 15 and 16, for example, a system 500 includes a holding device 540 that uses magnetic force to fix guide wire 170 relative to inner member 110. System 500 is similar to system 100 discussed above. However, holding device 540 includes a magnet 565 at the distal end of beam 145. Magnet 565 can be formed of one or more magnetic materials. Examples of magnetic materials include iron and cobalt. Any of various other magnetic materials can alternatively or additionally be used.

When spring 150 is expanded, as shown in FIG. 15, magnet 565 rests against an outer surface of inner member 110. The magnetic force produced by magnet 565 can help to prevent guide wire 170 from moving longitudinally within lumen 125 relative to inner member 110. Upon depressing button 155, which compresses spring 150, magnet 565 is moved away from inner member 110 and guide wire 170. As a result, the magnetic force acting on guide wire 170 is weaker and guide wire 170 can move longitudinally within lumen 125.

In certain embodiments, holding device 540 includes multiple magnets. The magnets can, for example, be arranged at spaced apart locations along the length of inner member 110. Alternatively or additionally, the magnets can be radially spaced about inner member 110.

As an alternative to or in addition to using one or more magnets to create a magnetic force about inner member 110, a conductive member (e.g., a copper wire) configured to carry an electrical current can be positioned around inner member 110. The conductive member can be electrically connected to a power source. During use of the system, the user can activate the power source to cause an electric current to flow through the conductive member, which can create a magnetic field around the inner member. This magnetic field can help to prevent the guide wire 170 from moving longitudinally relative to inner member 110.

While various embodiments have been described above, other embodiments are possible.

As an example, while many of the embodiments above describe systems that are generally in a locked configuration and can be unlocked by activating the holding device, the systems can alternatively be configured such that they are generally unlocked and can be locked by activating the holding device. Similarly, those systems that have been described as generally being in an unlocked configuration but being capable of being locked by activating the holding device can alternatively be configured such that they are generally in a locked configuration and are capable of being unlocked by activating the holding device.

As a further example, while the embodiments above describe a inner member having a relatively rigid region and a relatively compliant region, in some embodiments, substantially the entire inner member has substantially the same compliance (e.g., substantially the entire inner member is relatively compliant or substantially the entire inner member is relatively rigid).

As an additional example, while the embodiments above describe endoprosthesis 120 as a self-expanding stent, other types of endoprostheses can alternatively or additionally be used. In certain embodiments, for example, endoprosthesis 120 is a balloon-expandable stent. In such embodiments, the inner member on which endoprosthesis 120 is carried can be a balloon catheter. Other examples of endoprostheses include stent-grafts and filters (e.g., arterial filters and venous filters).

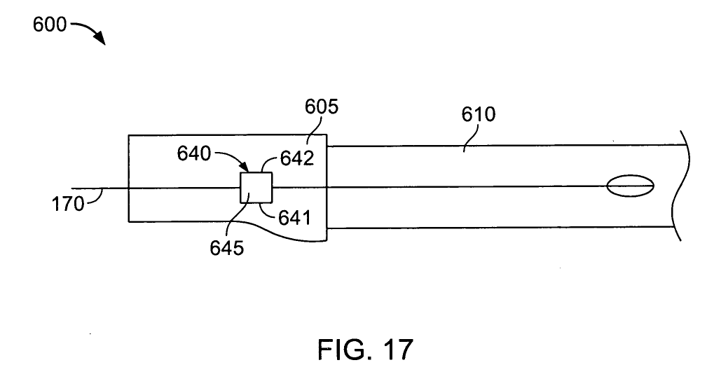

As another example, while over-the-wire systems have been described, other types of systems, such as rapid exchange systems, can alternatively or additionally be used. Referring to FIG. 17, for example, a system 600 includes a rapid exchange catheter 610. A handle 605 is secured to catheter 610, and includes a holding device 640. Holding device 640 includes a resilient tab 645 that is attached to a housing of handle 605 at its bottom edge 641 and is free at its top edge 642. Resilient tab 645 can be formed of one or more resilient materials such as silicones and thermoplastic rubbers.

During use of system 600, guide wire 170 can be inserted into a body vessel and then a distal portion of system 600 can be guided over guide wire 170 until positioned within a target region of the vessel. Unlike over-the-wire systems, guide wire 170 is not generally contained within handle 105. During use, however, guide wire 170 can be positioned between resilient tab 645 and handle 105. When no force is applied by the user to resilient tab 645, guide wire is free to move longitudinally. To fix guide wire 170 relative to handle 105 (and to catheter 610), the user can press (e.g., with his or her thumb) resilient tab 645 against guide wire 170 such that guide wire 170 is compressed between resilient tab 645 and handle 105. As an alternative to or in addition to resilient tab 645, any of the various other types of holding devices described herein can be used.

As a further example, while the systems of the embodiments above include endoprostheses, the systems can alternatively or additionally be equipped with other devices, such as laser ablation tools and inflatable balloons.

As an additional example, while the embodiments above show the systems being used in blood vessels of a subject, the systems can alternatively or additionally be used within any of various other body vessels or cavities of a subject. For example, the systems can be used within pulmonary vessels, gastrointestinal vessels, urinary vessels, reproductive vessels, biliary vessels, lymphatic vessels, the thoracic cavity (e.g., the heart, the lungs, the trachea, the esophagus, large blood vessels), the abdominal cavity (e.g., the gastrointestinal tract, the kidneys), the pelvic cavity (e.g., the urogenital system, the rectum), and the cranial cavity (e.g., the brain, vertebral canal).

Other embodiments are in the claims.

1. A system, comprising:
a catheter defining a lumen;
a handle secured to the catheter; and
a holding device secured to the handle, the holding device configured so that, when a guide wire is disposed in the lumen of the catheter, the holding device can hold the guide wire in a substantially fixed position relative to the catheter.

2. The system of claim 1, wherein the holding device is at least partially disposed within the handle.

3. The system of claim 1, wherein the holding device is secured to an end of the handle.

4. The system of claim 1, wherein the holding device is manually operable.

5. The system of claim 4, wherein the holding device comprises a button configured to be depressed by a user.

6. The system of claim 1, wherein the holding device comprises first and second positions, and
when the holding device is in the first position and a guide wire is disposed in the lumen of the catheter, the holding device holds the guide wire in a substantially fixed position relative to the catheter, and
when the holding device is in the second position and a guide wire is disposed in the lumen of the catheter, the guide wire is free to move longitudinally relative to the catheter.

7. The system of claim 1, wherein, when a guide wire is disposed in the lumen of the catheter, the holding device can bring the guide wire and the catheter into contact with one another.

8. The system of claim 7, wherein the holding device is adjacent an outer surface of the catheter.

9. The system of claim 7, wherein, when a guide wire is disposed in the lumen of the catheter, the holding device can bring the guide wire and an inner surface of the catheter into contact with one another.

10-11. (canceled)

12. The system of claim 1, wherein the catheter comprises a first region and a second region, the first region being more compliant than the second region.

13. The system of claim 12, wherein the holding device is configured to deform the first region of the catheter.

14. The system of claim 13, wherein, when the holding device deforms the first region of the catheter and a guide wire is disposed in the lumen of the catheter, the guide wire and the catheter are brought into contact with one another.

15-18. (canceled)

19. The system of claim 1, wherein the holding device comprises multiple members, and when a guide wire is disposed in the lumen of the catheter, the multiple members can bring the guide wire and the catheter into contact with one another.

20-21. (canceled)

22. The system of claim 1, wherein the holding device comprises at least one spring loaded member.

23. The system of claim 22, wherein the spring loaded member comprises a beam connected to a spring.

24-25. (canceled)

26. The system of claim 1, wherein the holding device comprises at least one clamp.

27. (canceled)

28. The system of claim 1, wherein the holding device comprises at least one inflatable member.

29-31. (canceled)

32. The system of claim 1, wherein the holding device comprises at least one magnet.

33. The system of claim 32, wherein the at least one magnet is attached to a beam.

34. The system of claim 33, wherein the beam is configured to move the magnet toward and away from the catheter.

35. The system of claim 1, wherein the holding device is positioned at a proximal region of the system.

36. The system of claim 1, wherein the handle is secured to a proximal region of the catheter.

37. The system of claim 1, further comprising an endoprosthesis carried by the catheter.

38. (canceled)

39. An implantable medical device system comprising:
an inner member defining a lumen;
an outer member configured so that an implantable medical device can be positioned between the inner member and the outer member;
a handle adapted to be secured to at least one of the inner and outer members; and
a holding device adapted to be secured to the handle, the holding device configured so that, when a guide wire is disposed in the lumen of the inner member, the holding device can hold the guide wire in a substantially fixed position relative to the inner member.

40. The system of claim 39, wherein the holding device is at least partially disposed within the handle.

41. The system of claim 39, wherein the holding device is manually operable.

42. The system of claim 39, wherein the holding device comprises a spring loaded member.

43. The system of claim 39, wherein the holding device comprises a clamp.

44. The system of claim 39, wherein the holding device comprises an inflatable member.

45. The system of claim 39, wherein the holding device comprises a magnet.

46. The system of claim 39, wherein the implantable medical device comprises an endoprosthesis.

47. A method of using a medical device comprising a handle secured to a catheter, the method comprising:
activating a holding device secured to the handle, the activated holding device securing an elongate element disposed within a lumen of the catheter to the catheter, a portion of the elongate element and the catheter being disposed within a body vessel of a subject.

48-54. (canceled)
